(19) United States
(12) Patent Application Publication
(10) Pub. No.: US 2020/0222670 A1
(43) Pub. Date: Jul. 16, 2020

(54) ANCHORING SYSTEMS AND RELATED METHODS

(71) Applicant: Stimwave Technologies Incorporated, Pompano Beach, FL (US)

(72) Inventors: Laura Tyler Perryman, Pompano Beach, FL (US); Chad David Andresen, Miami Beach, FL (US); Benjamin Speck, Boca Raton, FL (US)

(21) Appl. No.: 16/691,823

(22) Filed: Nov. 22, 2019

Related U.S. Application Data
(60) Provisional application No. 62/792,506, filed on Jan. 15, 2019.

(51) Int. Cl. A61M 25/04; A61M 25/00

(52) U.S. Cl. CPC A61M 25/04; A61M 2025/0293; A61M 25/0043

(57) ABSTRACT

A fixation device for securing a catheter to a tissue within a body includes a shaft defining a lumen configured to receive the catheter and multiple anchors disposed along the shaft. Each anchor of the multiple anchors includes multiple protrusive elements that are biased to an extended configuration in which the multiple protrusive elements extend radially from the shaft for engaging the tissue to secure the catheter to the tissue when the catheter is disposed within the lumen. The multiple protrusive elements can be adjusted from the extended configuration to a collapsed configuration in which the multiple protrusive elements are oriented parallel to the shaft.

### CROSS-REFERENCE TO RELATED APPLICATION

This application claims the benefit of U.S. Provisional Application No. 62/792,506, filed Jan. 15, 2019, and titled “Anchoring Systems and Related Methods,” which is incorporated by reference.

### TECHNICAL FIELD

This disclosure relates to anchoring systems used for securing implanted catheters to surrounding tissues within a body, such as within a subcutaneous space.

### BACKGROUND

Modulation of tissue within the body by electrical stimulation has become an important type of therapy for treating chronic, disabling conditions, such as chronic pain, problems of movement initiation and control, involuntary movements, dystonia, urinary and fecal incontinence, sexual difficulties, vascular insufficiency, and heart arrhythmia. For example, an external antenna can be used to send electrical energy to electrodes on an implanted tissue stimulator that can pass pulsatile electrical currents of controllable frequency, pulse width, and amplitudes to a tissue. In order to deliver a desired therapy to the tissue, the tissue stimulator should be optimally positioned with respect to the tissue in a secure manner.

### SUMMARY

In general, this disclosure relates to anchoring systems used for securing implanted catheters to surrounding tissues within a body, such as within a subcutaneous space. In some examples, the catheters form housings of tissue stimulators that are designed to deliver electrical therapy to the surrounding tissues.

In one aspect, a device for securing a catheter to a tissue within a body includes a shaft defining a lumen configured to receive the catheter and multiple anchors disposed along the shaft. Each anchor of the multiple anchors includes multiple protrusive elements that are biased to an extended configuration in which the multiple protrusive elements extend radially from the shaft for engaging the tissue to secure the catheter to the tissue when the catheter is disposed within the lumen. The multiple protrusive elements can be adjusted from the extended configuration to a collapsed configuration in which the multiple protrusive elements are oriented parallel to the shaft.

Embodiments may provide one or more of the following features.

In some embodiments, the lumen is sized such that the catheter can be secured to the shaft via a friction fit.

In some embodiments, the multiple anchors are integral with the shaft.

In some embodiments, the multiple anchors are axially spaced apart from each other by a distance of about 0.1 mm to about 5 mm.

In some embodiments, each anchor of the multiple anchors includes multiple hinges at which the multiple protrusive elements are pivotal with respect to the shaft.

In some embodiments, the multiple hinges include living hinges, elbow joints, or wire spring hinges.

In some embodiments, the multiple protrusive elements of each anchor of the multiple anchors extend in a proximal direction from the multiple hinges while the multiple protrusive elements are arranged in the extended configuration.

In some embodiments, the multiple protrusive elements of each anchor of the multiple anchors extends in a distal direction from the multiple hinges while the multiple protrusive elements are arranged in the collapsed configuration.

In some embodiments, the fixation device is made of one or more materials including polyurethane, silicone, carbothane, and elasthane.

In some embodiments, the fixation device has a durometer of 70 A to 60D.

In some embodiments, the multiple protrusive elements of each anchor of the multiple anchors is arranged about a circumference of the shaft.

In some embodiments, the protrusive elements of the multiple protrusive elements are equally spaced apart from each other about the circumference of the shaft.

In some embodiments, the multiple protrusive elements of each anchor of the multiple anchors are disposed substantially flush with the shaft while the multiple protrusive elements are arranged in the collapsed configuration.

In some embodiments, the shaft defines an interior profile along the lumen that includes protrusions and recessions.

In some embodiments, the interior profile of the shaft is complementary to an exterior profile of the catheter.

In some embodiments, the catheter includes a housing of an implantable tissue stimulator.

In some embodiments, the fixation device is an implantable device.

In some embodiments, the multiple protrusive elements of each anchor have a length of about 0.5 mm to about 5 mm.

In another aspect, an anchoring system for securing a catheter to a tissue within a body includes a fixation device and a deployment tool for assembling the fixation device with the catheter. The fixation device includes a shaft defining a lumen configured to receive the catheter and multiple anchors disposed along the shaft. Each anchor of the multiple anchors includes multiple protrusive elements that are biased to an extended configuration in which the multiple protrusive elements extend radially from the shaft for engaging the tissue to secure the catheter to the tissue when the catheter is disposed within the lumen. The multiple protrusive elements can be adjusted from the extended configuration to a collapsed configuration in which the multiple protrusive elements are oriented parallel to the shaft. The deployment tool includes a tubular housing configured to carry the fixation device, and the catheter secured therein, while the multiple protrusive elements are arranged in the collapsed configuration.

In another aspect, a method of using a device to secure a catheter to a tissue within a body includes placing the catheter within a fixation device carried by a tubular housing, and positioning the tubular housing, carrying the fixation device and the catheter therein, at a location adjacent to the tissue. The method further includes moving the fixation device distally out of the tubular housing at the location such that multiple protrusive elements of multiple anchors of the fixation device can expand from a collapsed configuration in which the multiple protrusive elements are oriented parallel to a shaft of the fixation device, to an extended configuration in which the multiple protrusive elements extend radially from the shaft to engage the tissue to secure the catheter to the tissue. The method further includes withdrawing the tubular housing from the fixation device at the location adjacent to the tissue.

### DESCRIPTION OF DRAWINGS

FIG. 1 is a perspective cross-sectional view of an anchoring system used to secure a catheter to surrounding tissue.

FIG. 2 is a perspective view of the anchoring system of FIG. 1 in a disassembled state.

FIG. 3 is an enlarged perspective view of an anchor of a fixation device of the anchoring system of FIG. 1.

FIG. 4 is an enlarged perspective view of a deployment tool partially assembled with the fixation device of the anchoring system of FIG. 1.

FIG. 5 is a flowchart of a method of using the anchoring system of FIG. 1 to secure a catheter to a tissue within a body.

FIG. 6 is a top view of various catheters that can be secured to a tissue with a fixation device of an anchoring system.

FIG. 7 illustrates side and front views of multiple protrusive elements of a fixation device of an anchoring system.

FIG. 8 illustrates front and perspective views of multiple protrusive elements of a fixation device of an anchoring system.

FIG. 9 illustrates side and front views of multiple protrusive elements of a fixation device of an anchoring system.

FIG. 10 is a side view of an anchoring system used to secure a catheter to surrounding tissue.

FIG. 11 is a system block diagram of a neural stimulation system including the catheter of FIG. 1 embodied as a housing of a tissue stimulator.

FIG. 12 is a detailed block diagram of the neural stimulation system of FIG. 11.

### DETAILED DESCRIPTION

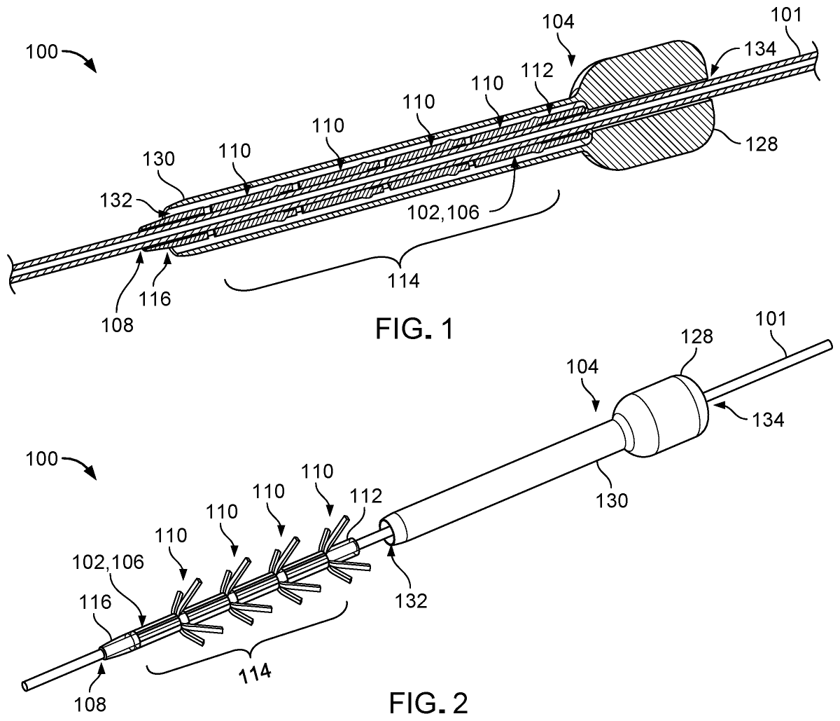

FIGS. 1 and 2 illustrate an anchoring system 100 that can be used to secure a catheter 101 to a tissue surrounding the catheter 101 within the body. The anchoring system 100 includes a fixation device 102 by which the catheter 101 can be attached to the tissue and a deployment tool 104 for assembling the fixation device 102 with the catheter 101.

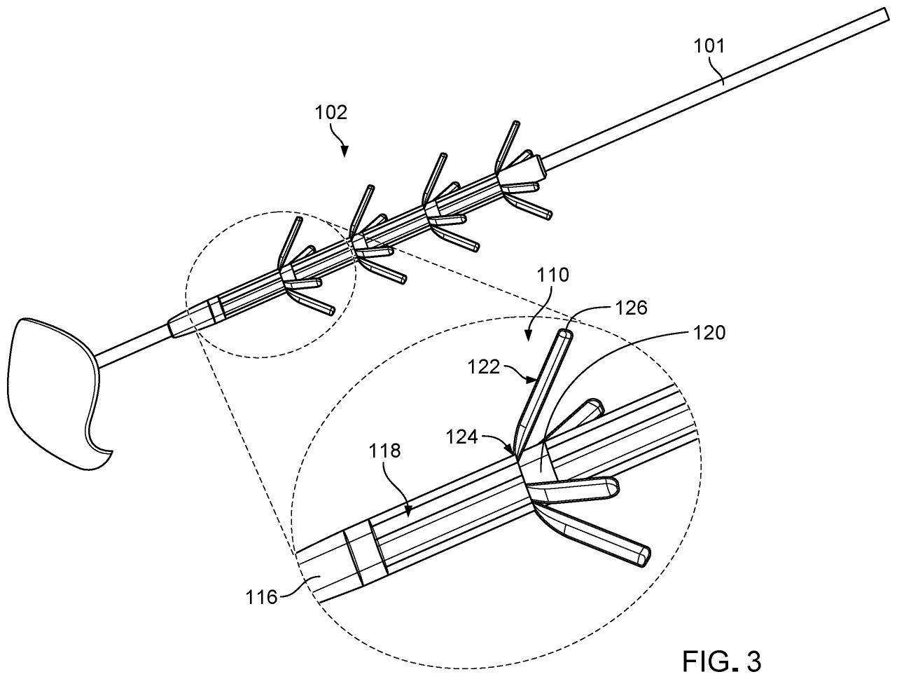

The fixation device 102 is an implantable device that includes a shaft 106 that defines a lumen 108 sized to receive the catheter 101 and multiple (e.g., four) anchors 110 disposed along the shaft 106. The shaft 106 has a generally tubular structure, and the lumen 108 accordingly has a generally circular cross-sectional shape. The shaft 106 includes a proximal portion 112 that can be seated within the deployment tool 104, a central portion 114 along which the anchors 110 are distributed, and a distal portion 116 that has a tapered exterior profile for facilitating distal movement of the fixation device 102 within the tissue and for preventing blunt trauma tissue damage during insertion. Referring to FIG. 3, the shaft 106 also defines multiple elongate openings 118 positioned about a circumference of the central portion 114 of the shaft 106 and multiple cylindrical wall sections 120 positioned at the anchors 110 along the central portion 114 of the shaft 106.

The anchors 110 are integrally formed with the shaft 106 and are reversibly adjustable between a collapsed configuration in which the anchors 110 are disposed within the deployment tool 104 (refer to FIG. 1) and an extended configuration in which the anchors 110 are not disposed within the deployment tool 104 or are otherwise unrestrained (refer to FIG. 2). Each anchor 110 includes multiple (e.g., four) protrusive elements 122 (e.g., tines) that are pivotable from the shaft 106 and respective hinges 124 (e.g., flexible living hinges) at which the protrusive elements 122 are pivotable. The protrusive elements 122 are aligned with the elongate openings 118 in the shaft 106 and are disposed within the elongate openings 118 such that the protrusive elements 122 are oriented parallel to the shaft 106 and are disposed substantially flush with the shaft 106 when the anchors 110 are constrained (e.g., compressed) to the collapsed configuration. The hinges 124 are located along distal ends of wall sections 120 of the shaft 106. Within an anchor 110, two protrusive elements 112 are located along opposite sides of the shaft 106 for securing the catheter 101 to the tissue along both sides of the catheter 101. The protrusive elements 122 are biased to the extended configuration shown in FIG. 2 in which distal ends 126 of the protrusive elements 112 are spaced apart radially from the shaft 106 and folded back proximally upon the hinges 124. The protrusive elements 112 have smooth surfaces to prevent tissue damage. Do the protrusive elements 112 are sized correctly to entangle with tissues for securement to the tissues.

The fixation device 102 is typically made of one or more biocompatible materials that are flexible enough to allow the shaft 106 to stretch (e.g., expand) slightly to accommodate the catheter 101 upon urging of the catheter 101 through the lumen 108 of the shaft 106 and to allow the protrusive elements 122 of the anchors 110 to repeatedly pivot at the hinges 124 without mechanical failure (e.g., tearing, fracturing, splitting, or otherwise separating) of the protrusive elements 122 or the hinges 124. Example materials from which the fixation device 102 is typically made include polyurethane, silicone, carbothane, and elasthane. Such materials may have a durometer in a range of 70 A to 60D.

The lumen 108 of the shaft 106 has a diameter that is slightly smaller than a diameter of the catheter 101, such when the catheter 101 is disposed within the lumen 108, the shaft 106 radially compresses the catheter 101, and the catheter 101 is secured to the shaft 106 via a friction fit. Accordingly, the catheter 101 is prevented from migrating with respect to the fixation device 102. The lumen 108 typically has a diameter of about 0.1 mm to about 1.8 mm. The shaft 106 typically has a length of about 5 mm to about 40 mm and a wall thickness of about 0.01 mm to about 0.2 mm. The cylindrical wall sections 120 typically have a length of about 0.1 mm to about 4 mm, which is sufficient to provide mechanical support for the hinges 124. The elongate openings 118 typically have a length of about 0.5 mm to about 5 mm and a width of about 0.1 mm to about 0.5 mm. The protrusive elements 122 of the anchors 110 typically have a length of about 0.5 mm to about 5 mm, a width of about 0.1 mm to about 0.5 mm, and a thickness of about 0.05 mm to about 0.2 mm. The anchors 110 are typically spaced apart from one another by about 0.1 mm to about 5 mm.

Referring again to FIGS. 1 and 2, the deployment tool 104 defines a handle 128 by which the deployment tool 104 can be manipulated and a tubular housing 130 that can carry the fixation device 102 with the catheter 101 disposed therein. Accordingly, the tubular housing 130 defines a receptacle 132 that is sized to receive the fixation device 102, and the handle 128 defines a channel 134 that is sized to receive the catheter 101. The deployment tool 104 is typically made of one or more materials, such as stainless steel, acrylonitrile butadiene styrene (ABS), polyethylene (PE), polypropylene (PP), or barium sulfate infused for radiopaque properties. The handle 128 typically has a length of about 5 mm to about 30 mm, an outer diameter of about 2.5 mm to about 10 mm, and an inner diameter (e.g., along the channel 134) of about 0.15 mm to about 2.0 mm. The tubular housing 130 typically has a length of about 5 mm to about 50 mm, an outer diameter of about 1.5 mm to about 5 mm, and an inner diameter (e.g., along the receptacle 132) of about 0.2 mm to about 2.5 mm.

In order to secure the catheter 101 to the tissue using the anchoring system 100, an incision is made adjacent an implantation site (e.g., a subcutaneous space) of the catheter 101, and the deployment tool 104, carrying the fixation device 102, is placed over the catheter 101, as shown in FIG. 1. The anchoring system 100, now carrying the catheter 101, is positioned using physiological landmarks at a location determined to be optimal for fixating to tissues with respect to the placement of the catheter 101 with respect to the tissue. The fixation device 102 is designed to be deployed in strong, fibrous tissue (e.g., ligaments, muscle, fascia, etc.) to optimize the amount of fixation. Before deployment it is beneficial to check that the fixation device 102 will be positioned at the correct tissue location. An imaging technique (e.g., x-ray or ultrasound) is then performed to confirm that a position of the catheter 101 is acceptable. Once an acceptable position is confirmed, a plunger is used to urge the catheter 101, with the fixation device 102 carried thereon, distally out of the deployment tool 104. The fixation device 102 is pushed along the catheter 101 using the deployment tool 104. When the user desires to release the fixation device 102, the deployment tool 104 is retracted in the proximal direction, leaving the fixation device 102 fixated to the catheter 101 in place.

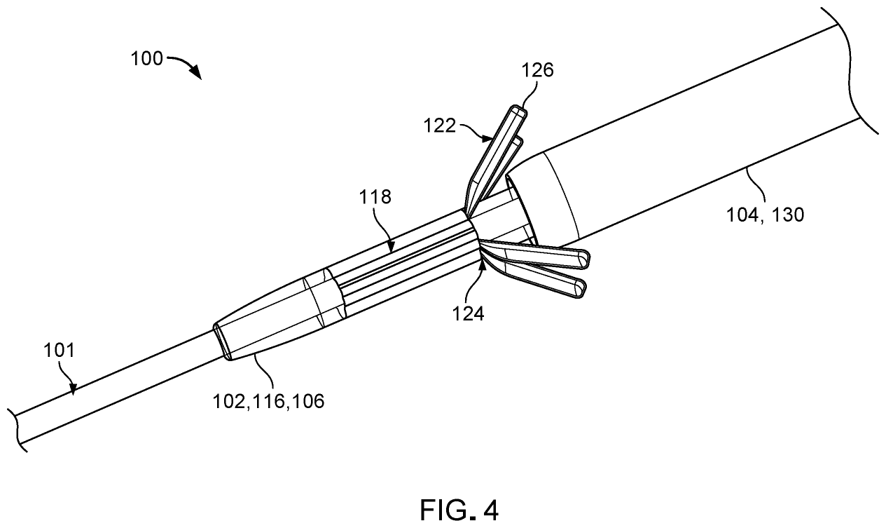

Referring to FIG. 4, as each anchor 110 is moved distally out of the receptacle 132 of the deployment tool 104, the protrusive elements 122 of the anchor 110 pivot (e.g., spring) radially outward in a proximal direction to their biased positions of the extended configuration of the anchor 110. In the extended configuration, the protrusive elements 122 exert a radial force on the tissue surrounding the catheter 101, thereby fixating the tissue to fix the catheter 101 (e.g., held within the fixation device 102 via a friction fit) in position with respect to the tissue. The catheter 101, with the fixation device 102 carried thereon, is pushed further distally until the catheter 101 and the fixation device 102 are completely removed from the deployment tool 104 such that all of the anchors 110 are disposed in the extended configuration to securely fix the catheter 101 to the tissue. The fixation device 102 can be gently pulled (e.g., tugged) to confirm that the protrusive elements 122 are secured to the tissue. The deployment tool 104 is then withdrawn from the implantation site through the incision and is either discarded or reloaded again with another fixation device 102.

If the catheter 101 needs to be removed from the body or needs to be repositioned within the body, another deployment tool 104 can be moved distally over the fixation device 102, with the catheter 101 secured therein, until the fixation device 102 is fully seated within the receptacle 132 of the deployment tool 104 with the anchors 110 in the collapsed configuration, as shown in FIG. 1. The tissue may sustain minimal damage during removal of the fixation elements from tissue. The deployment tool 104, now carrying the fixation device 102 and the catheter 101, can then be removed from the body or moved in any direction to be repositioned within the body and subsequently removed from the fixation device 102 to secure the catheter 101 to the tissue at a different location.

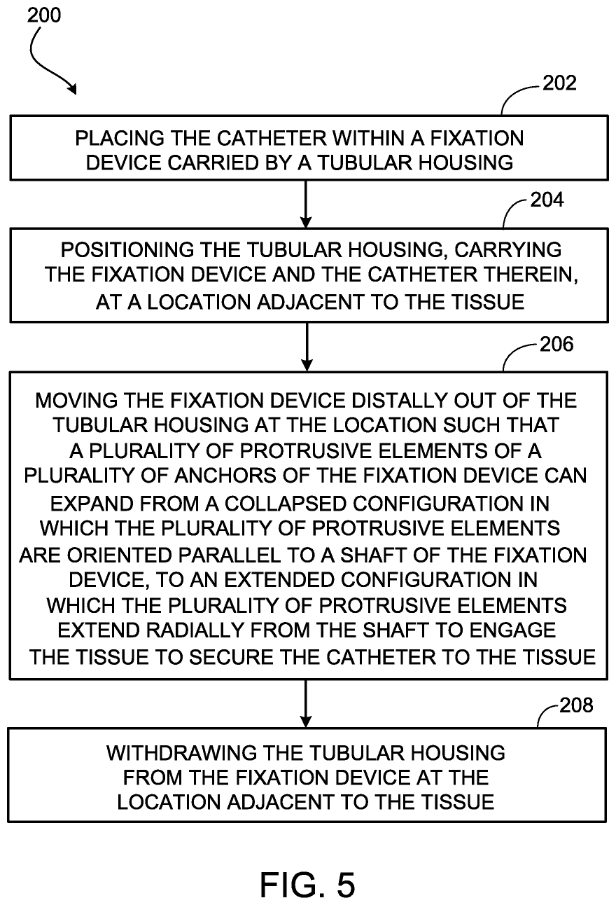

FIG. 5 provides a flowchart that illustrates a method 200 of using an anchoring system (e.g., the anchoring system 100) to secure a catheter (e.g., the catheter 101) to a tissue within a body. In some examples, the method includes placing the catheter within a fixation device (e.g., the fixation device 102) carried by a tubular housing (e.g., the tubular housing 130) (202). In some examples the method further includes positioning the tubular housing, carrying the fixation device and the catheter therein, at a location adjacent to the tissue (204). In some examples, the method further includes moving the fixation device distally out of the tubular housing at the location such that multiple protrusive elements (e.g., the protrusive elements 122) of multiple anchors (e.g., the anchors 110) of the fixation device can expand from a collapsed configuration in which the multiple protrusive elements are oriented parallel to a shaft (e.g., the shaft 106) of the fixation device, to an extended configuration in which the multiple protrusive elements extend radially from the shaft to engage the tissue to secure the catheter to the tissue (206). In some examples, the method further includes withdrawing the tubular housing from the fixation device at the location adjacent to the tissue (208).

While the anchoring system 100 has been described and illustrated as including certain dimensions, sizes, shapes, materials, arrangements, and configurations, in some embodiments, anchoring systems that are similar in structure and function to either of the anchoring system 100 may include different dimensions, sizes, shapes, materials, arrangements, or configurations. For example, while the fixation device 102 of the anchoring system 100 has been described and illustrated as including a lumen 108 with a circular cross-sectional shape that is sized to grip the catheter 101 via a friction fit, in some embodiments, a fixation device includes an interior surface that is complementary to an exterior surface of a catheter for locking the catheter in place within the fixation device.

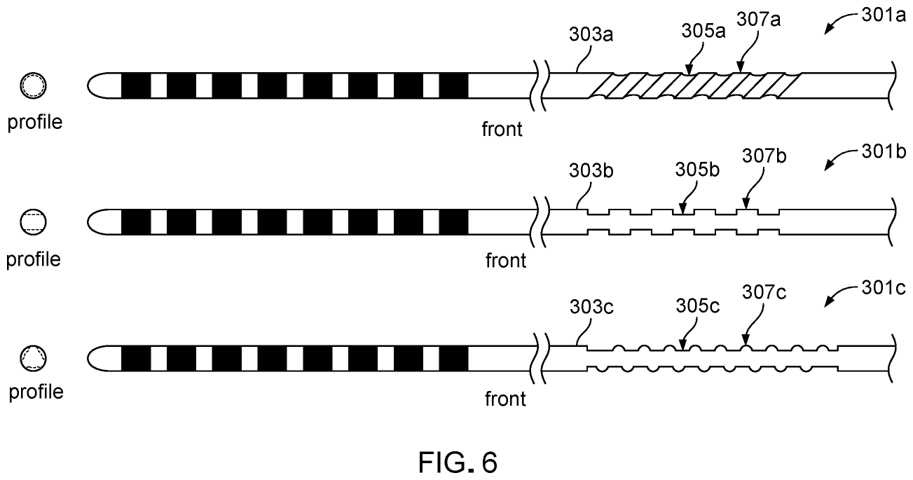

Referring to FIG. 6, an anchoring system can include the deployment tool 104 (shown in FIGS. 1 and 2) and a fixation device that is substantially similar in construction and function to the fixation device 102, except that a shaft of the fixation device has an interior surface that is complementary to an exterior surface 303a, 303b, or 303c of a catheter 301a, 301b, or 301c. For example, the interior surface of the fixation device may define protrusions and recessions that respectively mate with recessions 305a, 305b, or 305c and protrusions 307a, 307b, or 307c of the exterior surface 303a, 303b, or 303c of the catheter 301a, 301b, or 301c. A mating between the interior surface of the fixation device and the complementary surface 303a, 303b, or 303c and a frictional fit between the interior surface and the complementary surface 303a, 303b, or 303c can together fix a position of the catheter 301a, 301b, or 301c with respect to the fixation device.

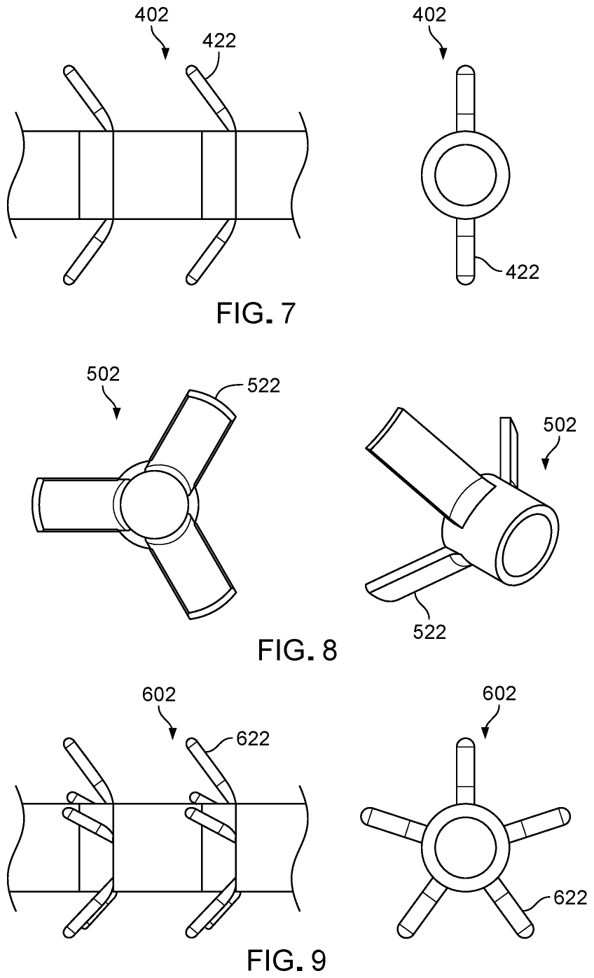

While the fixation device 102 of the anchoring system 100 has been described and illustrated as including anchors 110 with four protrusive elements 122, in some embodiments, an anchoring system includes a fixation device that has anchors with a different number of protrusive elements. For example, FIGS. 7-9 respectively illustrate fixation devices 402, 502, 602 that include two protrusive elements 422, three protrusive elements 522, and five protrusive elements 622 that are equally spaced about a circumference of shafts 406, 506, 606. The fixation devices 402, 502, 602 are otherwise substantially similar in construction and function to the fixation device 102 and are accordingly designed to be used with the deployment tool 104. In various embodiments, the protrusive elements 122, 422, 522, 622 may be equally or unequally spaced about a circumference of the fixation devices 102, 402, 502, 602.

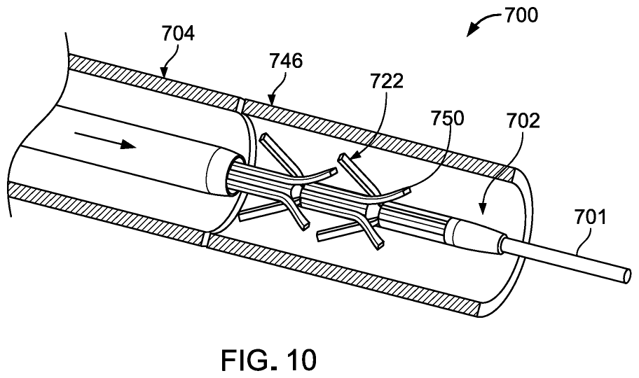

While the fixation device 102 of the anchoring system 100 has been described and illustrated as including anchors 110 with protrusive elements 122 that open in the same direction, in some embodiments, an anchoring system includes a fixation device that has anchors with protrusive elements that open in more than one direction. For example, FIG. 10 illustrates an anchoring system 700 including a deployment tool 704 and a fixation device 702 that includes protrusive elements 722 that open in the proximal direction and protrusive elements 750 that open in the distal direction. A sleeve 746 surrounds the fixation device 702 to maintain the protrusive elements 722, 750 in a collapsed configuration and can be removed to allow the protrusive elements 722, 750 to be released to the extended configuration to secure a catheter 701 carried therein to a surrounding tissue. The fixation device 702 is otherwise similar in construction and function to the fixation device 102. Owing to the opposing directions in which the protrusive elements 722, 750 project into the tissue, the fixation device 702 may function as a permanent implant that cannot be retrieved (e.g., or easily retrieved without damaging the tissue).

While the fixation device 102 of the anchoring system 100 has been described and illustrated as including the living hinges 124, in some embodiments, an anchoring system includes a fixation device that has a different type of hinge, such as elbow joints or wire spring hinges.

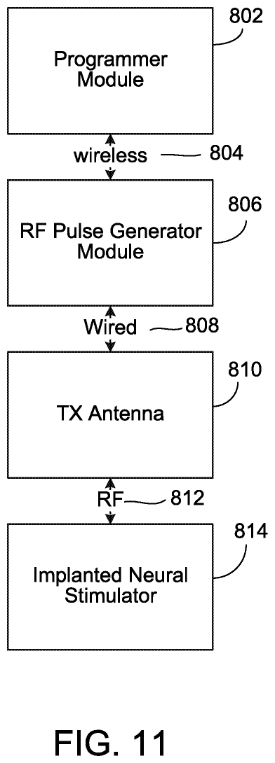

In some embodiments, the catheter 101 (e.g., or any other above-mentioned catheter) is embodied as a housing of a tissue stimulator 814 (e.g., a wireless, implantable tissue stimulator) that may be provided as part of a tissue stimulation system, such as a neural stimulation system 800. Referring to FIG. 11, the neural stimulation system 800 is designed to send electrical pulses to a nearby (e.g., adjacent or surrounding) target nerve tissue to stimulate the target nerve tissue by using remote radio frequency (RF) energy without cables and without inductive coupling to power the tissue stimulator 814. Accordingly, the tissue stimulator 814 is provided as a passive tissue stimulator in the neural stimulation system 800. In some examples, the target nerve tissue is in the spinal column and may include one or more of the spinothalamic tracts, the dorsal horn, the dorsal root ganglia, the dorsal roots, the dorsal column fibers, and the peripheral nerves bundles leaving the dorsal column or the brainstem. In some examples, the target nerve tissue may include one or more of cranial nerves, abdominal nerves, thoracic nerves, trigeminal ganglia nerves, nerve bundles of the cerebral cortex, deep brain, sensory nerves, and motor nerves.

The neural stimulation system 800 further includes a programmer module 802, an RF pulse generator module 806 (e.g., a controller module), and a transmit (TX) antenna 810. In some embodiments, the programmer module 802 is a computing device (e.g., a smart phone, another mobile computing device, or a stationary computing device) running a software application that supports a wireless connection 804 (e.g., via Bluetooth). The software application can enable the user to view a system status and system diagnostics, change various parameters, increase and decrease a desired stimulus amplitude of the electrical pulses, and adjust a feedback sensitivity of the RF pulse generator module 806, among other functions.

The RF pulse generator module 806 includes stimulation circuitry, a battery to power generator electronics, and communication electronics that support the wireless connection 804. In some embodiments, the RF pulse generator module 806 is designed to be worn external to the body, and the TX antenna 810 (e.g., located external to the body) is connected to the RF pulse generator module 806 by a wired connection 808. Accordingly, the RF pulse generator module 806 and the TX antenna 810 may be incorporated into a wearable accessory (e.g., a belt or a harness design) or a clothing article such that electric radiative coupling can occur through the skin and underlying tissue to transfer power and/or control parameters to the tissue stimulator 814.

The TX antenna 810 can be coupled directly to tissues within the body to create an electric field that powers the implanted tissue stimulator 814. The TX antenna 810 communicates with the tissue stimulator 814 through an RF interface. For instance, the TX antenna 810 radiates an RF transmission signal that is modulated and encoded by the RF pulse generator module 806. The tissue stimulator 814 includes one or more antennas (e.g., dipole antennas) that can receive and transmit through an RF interface 812. In particular, the coupling mechanism between the TX antenna 810 and the one or more antennas on the tissue stimulator 814 is electrical radiative coupling and not inductive coupling. In other words, the coupling is through an electric field rather than through a magnetic field. Through this electrical radiative coupling, the TX antenna 810 can provide an input signal to the tissue stimulator 814.

In addition to the one or more antennas, the tissue stimulator 814 further includes internal receiver circuit components that can capture the energy carried by the input signal sent from the TX antenna 804 and demodulate the input signal to convert the energy to an electrical waveform. The receiver circuit components can further modify the waveform to create electrical pulses suitable for stimulating the target neural tissue. The tissue stimulator 814 further includes electrodes that can deliver the electrical pulses to the target neural tissue. For example, the circuit components may include wave conditioning circuitry that rectifies the received RF signal (e.g., using a diode rectifier), transforms the RF energy to a low frequency signal suitable for the stimulation of neural tissue, and presents the resulting waveform to an array of the electrodes. In some implementations, the power level of the input signal directly determines an amplitude (e.g., a power, a current, and/or a voltage) of the electrical pulses applied to the target neural tissue by the electrodes. For example, the input signal may include information encoding stimulus waveforms to be applied at the electrodes.

In some implementations, the RF pulse generator module 806 can remotely control stimulus parameters of the electrical pulses applied to the target neural tissue by the electrodes and monitor feedback from the tissue stimulator 814 based on RF signals received from the tissue stimulator 814. For example, a feedback detection algorithm implemented by the RF pulse generator module 806 can monitor data sent wirelessly from the tissue stimulator 814, including information about the energy that the tissue stimulator 814 is receiving from the RF pulse generator 806 and information about the stimulus waveform being delivered to the electrodes. Accordingly, the circuit components internal to the tissue stimulator 814 may also include circuitry for communicating information back to the RF pulse generator module 806 to facilitate the feedback control mechanism. For example, the tissue stimulator 814 may send to the RF pulse generator module 806 a stimulus feedback signal that is indicative of parameters of the electrical pulses, and the RF pulse generator module 806 may employ the stimulus feedback signal to adjust parameters of the signal sent to the tissue stimulator 814.

In order to provide an effective therapy for a given medical condition, the neural stimulation system 800 can be tuned to provide the optimal amount of excitation or inhibition to the nerve fibers by electrical stimulation. A closed loop feedback control method can be used in which the output signals from the tissue stimulator 814 are monitored and used to determine the appropriate level of neural stimulation current for maintaining effective neuronal activation. Alternatively, in some cases, the patient can manually adjust the output signals in an open loop control method.

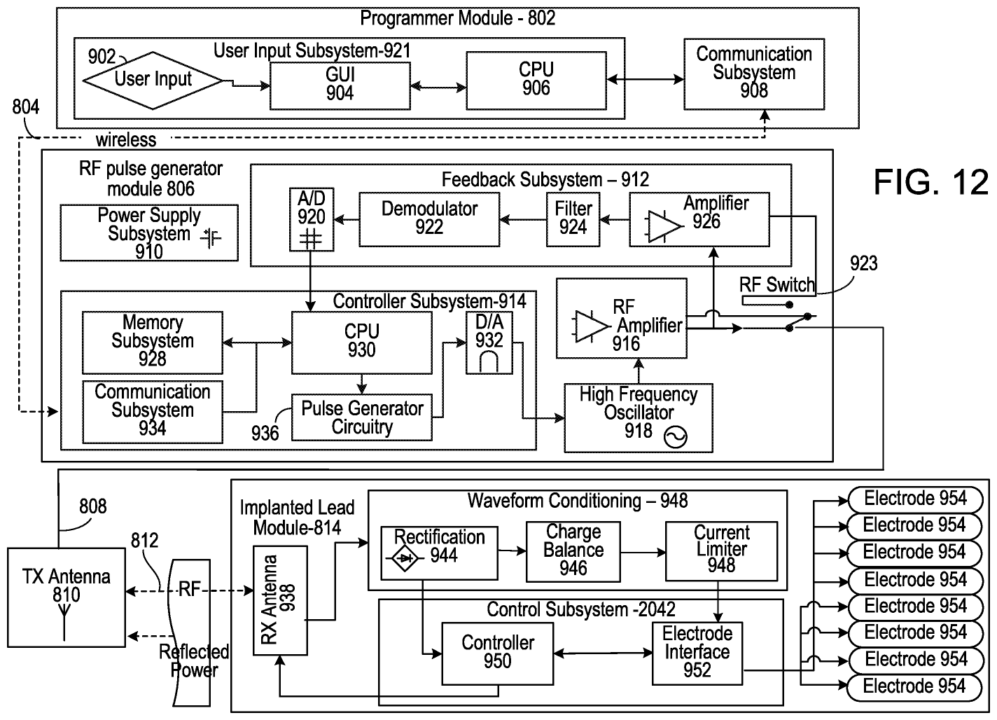

FIG. 12 depicts a detailed diagram of the neural stimulation system 800. The programmer module 802 may be used as a vehicle to handle touchscreen input on a graphical user interface (GUI) 904 and may include a central processing unit (CPU) 906 for processing and storing data. The programmer module 802 includes a user input system 921 and a communication subsystem 908. The user input system 921 can allow a user to input or adjust instruction sets in order to adjust various parameter settings (e.g., in some cases, in an open loop fashion). The communication subsystem 908 can transmit these instruction sets (e.g., and other information) via the wireless connection 804 (e.g., via a Bluetooth or Wi-Fi connection) to the RF pulse generator module 806. The communication subsystem 908 can also receive data from RF pulse generator module 806.

The programmer module 802 can be utilized by multiple types of users (e.g., patients and others), such that the programmer module 802 may serve as a patient's control unit or a clinician's programmer unit. The programmer module 802 can be used to send stimulation parameters to the RF pulse generator module 806. The stimulation parameters that can be controlled may include a pulse amplitude in a range of 0 mA to 20 mA, a pulse frequency in a range of 0 Hz to 2000 Hz, and a pulse width in a range of 0 ms to 2 ms. In this context, the term pulse refers to the phase of the waveform that directly produces stimulation of the tissue. Parameters of a charge-balancing phase (described below) of the waveform can similarly be controlled. The user can also optionally control an overall duration and a pattern of a treatment.

The tissue stimulator 814 or the RF pulse generator module 806 may be initially programmed to meet specific parameter settings for each individual patient during an initial implantation procedure. Because medical conditions or the body itself can change over time, the ability to readjust the parameter settings may be beneficial to ensure ongoing efficacy of the neural modulation therapy.

Signals sent by the RF pulse generator module 806 to the tissue stimulator 814 may include both power and parameter attributes related to the stimulus waveform, amplitude, pulse width, and frequency. The RF pulse generator module 806 can also function as a wireless receiving unit that receives feedback signals from the tissue stimulator 814. To that end, the RF pulse generator module 806 includes microelectronics or other circuitry to handle the generation of the signals transmitted to the tissue stimulator 814, as well as feedback signals received from tissue stimulator 814. For example, the RF pulse generator module 806 includes a controller subsystem 914, a high-frequency oscillator 918, an RF amplifier 916, an RF switch, and a feedback subsystem 912.

The controller subsystem 914 includes a CPU 930 to handle data processing, a memory subsystem 928 (e.g., a local memory), a communication subsystem 934 to communicate with the programmer module 802 (e.g., including receiving stimulation parameters from the programmer module 802), pulse generator circuitry 936, and digital/analog (D/A) converters 932.

The controller subsystem 914 may be used by the user to control the stimulation parameter settings (e.g., by controlling the parameters of the signal sent from RF pulse generator module 806 to tissue stimulator 814). These parameter settings can affect the power, current level, or shape of the electrical pulses that will be applied by the electrodes. The programming of the stimulation parameters can be performed using the programming module 802 as described above to set a repetition rate, pulse width, amplitude, and waveform that will be transmitted by RF energy to a receive (RX) antenna 938 (e.g., or multiple RX antennas 938) within the tissue stimulator 814. The RX antenna 938 may be a dipole antenna or another type of antenna. A clinician user may have the option of locking and/or hiding certain settings within a programmer interface to limit an ability of a patient user to view or adjust certain parameters since adjustment of certain parameters may require detailed medical knowledge of neurophysiology, neuroanatomy, protocols for neural modulation, and safety limits of electrical stimulation.

The controller subsystem 914 may store received parameter settings in the local memory subsystem 928 until the parameter settings are modified by new input data received from the programmer module 802. The CPU 906 may use the parameters stored in the local memory to control the pulse generator circuitry 936 to generate a stimulus waveform that is modulated by the high frequency oscillator 918 in a range of 300 MHz to 8 GHz. The resulting RF signal may then be amplified by an RF amplifier 926 and sent through an RF switch 923 to the TX antenna 810 to reach the RX antenna 938 through a depth of tissue.

In some implementations, the RF signal sent by the TX antenna 810 may simply be a power transmission signal used by tissue stimulator 814 to generate electric pulses. In other implementations, the RF signal sent by the TX antenna 810 may be a telemetry signal that provides instructions about various operations of the tissue stimulator 814. The telemetry signal may be sent by the modulation of the carrier signal through the skin. The telemetry signal is used to modulate the carrier signal (e.g., a high frequency signal) that is coupled to the antenna 938 and does not interfere with the input received on the same lead to power the tissue stimulator 814. In some embodiments, the telemetry signal and the powering signal are combined into one signal, where the RF telemetry signal is used to modulate the RF powering signal such that the tissue stimulator 814 is powered directly by the received telemetry signal. Separate subsystems in the tissue stimulator 814 harness the power contained in the signal and interpret the data content of the signal.

The RF switch 923 may be a multipurpose device (e.g., a dual directional coupler) that passes the relatively high amplitude, extremely short duration RF pulse to the TX antenna 810 with minimal insertion loss, while simultaneously providing two low-level outputs to the feedback subsystem 912. One output delivers a forward power signal to the feedback subsystem 912, where the forward power signal is an attenuated version of the RF pulse sent to the TX antenna 810, and the other output delivers a reverse power signal to a different port of the feedback subsystem 912, where reverse power is an attenuated version of the reflected RF energy from the TX Antenna 810.

During the on-cycle time (e.g., while an RF signal is being transmitted to tissue stimulator 814), the RF switch 923 is set to send the forward power signal to feedback subsystem 912. During the off-cycle time (e.g., while an RF signal is not being transmitted to the tissue stimulator 814), the RF switch 923 can change to a receiving mode in which the reflected RF energy and/or RF signals from the tissue stimulator 814 are received to be analyzed in the feedback subsystem 912.

The feedback subsystem 912 of the RF pulse generator module 806 may include reception circuitry to receive and extract telemetry or other feedback signals from tissue stimulator 814 and/or reflected RF energy from the signal sent by TX antenna 810. The feedback subsystem 912 may include an amplifier 926, a filter 924, a demodulator 922, and an A/D converter 920. The feedback subsystem 912 receives the forward power signal and converts this high-frequency AC signal to a DC level that can be sampled and sent to the controller subsystem 914. In this way, the characteristics of the generated RF pulse can be compared to a reference signal within the controller subsystem 914. If a disparity (e.g., an error) exists in any parameter, the controller subsystem 914 can adjust the output to the RF pulse generator 806. The nature of the adjustment can be proportional to the computed error. The controller subsystem 914 can incorporate additional inputs and limits on its adjustment scheme, such as the signal amplitude of the reverse power and any predetermined maximum or minimum values for various pulse parameters.

The reverse power signal can be used to detect fault conditions in the RF-power delivery system. In an ideal condition, when TX antenna 810 has perfectly matched impedance to the tissue that it contacts, the electromagnetic waves generated from the RF pulse generator module 806 pass unimpeded from the TX antenna 810 into the body tissue. However, in real-world applications, a large degree of variability exists in the body types of users, types of clothing worn, and positioning of the antenna 810 relative to the body surface. Since the impedance of the antenna 810 depends on the relative permittivity of the underlying tissue and any intervening materials and on an overall separation distance of the antenna 810 from the skin, there can be an impedance mismatch at the interface of the TX antenna 810 with the body surface in any given application. When such a mismatch occurs, the electromagnetic waves sent from the RF pulse generator module 806 are partially reflected at this interface, and this reflected energy propagates backward through the antenna feed.

The dual directional coupler RF switch 923 may prevent the reflected RF energy propagating back into the amplifier 926, and may attenuate this reflected RF signal and send the attenuated signal as the reverse power signal to the feedback subsystem 912. The feedback subsystem 912 can convert this high-frequency AC signal to a DC level that can be sampled and sent to the controller subsystem 914. The controller subsystem 914 can then calculate the ratio of the amplitude of the reverse power signal to the amplitude of the forward power signal. The ratio of the amplitude of reverse power signal to the amplitude level of forward power may indicate severity of the impedance mismatch.

In order to sense impedance mismatch conditions, the controller subsystem 914 can measure the reflected-power ratio in real time, and according to preset thresholds for this measurement, the controller subsystem 914 can modify the level of RF power generated by the RF pulse generator module 806. For example, for a moderate degree of reflected power the course of action can be for the controller subsystem 914 to increase the amplitude of RF power sent to the TX antenna 810, as would be needed to compensate for slightly non-optimum but acceptable TX antenna coupling to the body. For higher ratios of reflected power, the course of action can be to prevent operation of the RF pulse generator module 806 and set a fault code to indicate that the TX antenna 810 has little or no coupling with the body. This type of reflected power fault condition can also be generated by a poor or broken connection to the TX antenna 810. In either case, it may be desirable to stop RF transmission when the reflected power ratio is above a defined threshold, because internally reflected power can lead to unwanted heating of internal components, and this fault condition means that the system cannot deliver sufficient power to the tissue stimulator 814 and thus cannot deliver therapy to the user.

The controller 942 of the tissue stimulator 814 may transmit informational signals, such as a telemetry signal, through the RX antenna 538 to communicate with the RF pulse generator module 806 during its receive cycle. For example, the telemetry signal from the tissue stimulator 814 may be coupled to the modulated signal on the RX antenna 938, during the on and off state of the transistor circuit to enable or disable a waveform that produces the corresponding RF bursts necessary to transmit to the external (or remotely implanted) pulse generator module 806. The RX antenna 938 may be connected to electrodes 954 in contact with tissue to provide a return path for the transmitted signal. An A/D converter can be used to transfer stored data to a serialized pattern that can be transmitted on the pulse modulated signal from the RX antenna 938 of the tissue stimulator 814.

A telemetry signal from the tissue stimulator 814 may include stimulus parameters, such as the power or the amplitude of the current that is delivered to the tissue from the electrodes 954. The feedback signal can be transmitted to the RF pulse generator module 806 to indicate the strength of the stimulus at the target nerve tissue by means of coupling the signal to the RX antenna 938, which radiates the telemetry signal to the RF pulse generator module 806. The feedback signal can include either or both an analog and digital telemetry pulse modulated carrier signal. Data such as stimulation pulse parameters and measured characteristics of stimulator performance can be stored in an internal memory device within the tissue stimulator 814 and sent on the telemetry signal. The frequency of the carrier signal may be in a range of 300 MHz to 8 GHz.

In the feedback subsystem 912, the telemetry signal can be down modulated using the demodulator 922 and digitized by being processed through the analog to digital (A/D) converter 920. The digital telemetry signal may then be routed to the CPU 930 with embedded code, with the option to reprogram, to translate the signal into a corresponding current measurement in the tissue based on the amplitude of the received signal. The CPU 930 of the controller subsystem 914 can compare the reported stimulus parameters to those held in local memory 928 to verify that the tissue stimulator 814 delivered the specified stimuli to target nerve tissue. For example, if the tissue stimulator 814 reports a lower current than was specified, the power level from the RF pulse generator module 806 can be increased so that the tissue stimulator 814 will have more available power for stimulation. The tissue stimulator 814 can generate telemetry data in real time (e.g., at a rate of 8 kbits per second). All feedback data received from the tissue stimulator 814 can be logged against time and sampled to be stored for retrieval to a remote monitoring system accessible by a health care professional for trending and statistical correlations.

The sequence of remotely programmable RF signals received by the RX antenna 938 may be conditioned into waveforms that are controlled within the tissue stimulator 814 by the control subsystem 942 and routed to the appropriate electrodes 954 that are located in proximity to the target nerve tissue. For instance, the RF signal transmitted from the RF pulse generator module 806 may be received by RX antenna 938 and processed by circuitry, such as waveform conditioning circuitry 940, within the tissue stimulator 814 to be converted into electrical pulses applied to the electrodes 954 through an electrode interface 952. In some implementations, the tissue stimulator 814 includes between two to sixteen electrodes 954.

The waveform conditioning circuitry 940 may include a rectifier 944, which rectifies the signal received by the RX antenna 938. The rectified signal may be fed to the controller 942 for receiving encoded instructions from the RF pulse generator module 806. The rectifier signal may also be fed to a charge balance component 946 that is configured to create one or more electrical pulses such that the one or more electrical pulses result in a substantially zero net charge at the one or more electrodes 954 (that is, the pulses are charge balanced). The charge balanced pulses are passed through the current limiter 948 to the electrode interface 952, which applies the pulses to the electrodes 954 as appropriate.

The current limiter 948 insures the current level of the pulses applied to the electrodes 954 is not above a threshold current level. In some implementations, an amplitude (for example, a current level, a voltage level, or a power level) of the received RF pulse directly determines the amplitude of the stimulus. In this case, it may be particularly beneficial to include current limiter 948 to prevent excessive current or charge being delivered through the electrodes 954, although the current limiter 548 may be used in other implementations where this is not the case. Generally, for a given electrode 954 having several square millimeters of surface area, it is the charge per phase that should be limited for safety (where the charge delivered by a stimulus phase is the integral of the current). But, in some cases, the limit can instead be placed on the current, where the maximum current multiplied by the maximum possible pulse duration is less than or equal to the maximum safe charge. More generally, the current limiter 948 acts as a charge limiter that limits a characteristic (for example, a current or duration) of the electrical pulses so that the charge per phase remains below a threshold level (typically, a safe-charge limit).

In the event the tissue stimulator 814 receives a “strong” pulse of RF power sufficient to generate a stimulus that would exceed the predetermined safe-charge limit, the current limiter 948 can automatically limit or “clip” the stimulus phase to maintain the total charge of the phase within the safety limit. The current limiter 948 may be a passive current limiting component that cuts the signal to the electrodes 954 once the safe current limit (the threshold current level) is reached. Alternatively, or additionally, the current limiter 948 may communicate with the electrode interface 952 to turn off all electrodes 954 to prevent tissue damaging current levels.

A clipping event may trigger a current limiter feedback control mode. The action of clipping may cause the controller to send a threshold power data signal to the RF pulse generator module 806. The feedback subsystem 912 detects the threshold power signal and demodulates the signal into data that is communicated to the controller subsystem 914. The controller subsystem 914 algorithms may act on this current-limiting condition by specifically reducing the RF power generated by the RF pulse generator module 806, or cutting the power completely. In this way, the RF pulse generator module 806 can reduce the RF power delivered to the body if the tissue stimulator 814 reports that it is receiving excess RF power.

The controller 950 may communicate with the electrode interface 952 to control various aspects of the electrode setup and pulses applied to the electrodes 954. The electrode interface 952 may act as a multiplex and control the polarity and switching of each of the electrodes 954. For instance, in some implementations, the tissue stimulator 814 has multiple electrodes 954 in contact with the target neural tissue, and for a given stimulus, the RF pulse generator module 806 can arbitrarily assign one or more electrodes to act as a stimulating electrode, to act as a return electrode, or to be inactive by communication of assignment sent wirelessly with the parameter instructions, which the controller 950 uses to set electrode interface 952 as appropriate. It may be physiologically advantageous to assign, for example, one or two electrodes 954 as stimulating electrodes and to assign all remaining electrodes 954 as return electrodes.

Also, in some implementations, for a given stimulus pulse, the controller 950 may control the electrode interface 952 to divide the current arbitrarily (or according to instructions from the RF pulse generator module 806) among the designated stimulating electrodes. This control over electrode assignment and current control can be advantageous because in practice the electrodes 954 may be spatially distributed along various neural structures, and through strategic selection of the stimulating electrode location and the proportion of current specified for each location, the aggregate current distribution on the target neural tissue can be modified to selectively activate specific neural targets. This strategy of current steering can improve the therapeutic effect for the patient.

In another implementation, the time course of stimuli may be arbitrarily manipulated. A given stimulus waveform may be initiated at a time T_start and terminated at a time T final, and this time course may be synchronized across all stimulating and return electrodes. Furthermore, the frequency of repetition of this stimulus cycle may be synchronous for all of the electrodes 954. However, the controller 950, on its own or in response to instructions from the RF pulse generator module 806, can control electrode interface 952 to designate one or more subsets of electrodes to deliver stimulus waveforms with non-synchronous start and stop times, and the frequency of repetition of each stimulus cycle can be arbitrarily and independently specified.

For example, a tissue stimulator 814 having eight electrodes 954 may be configured to have a subset of five electrodes, called set A, and a subset of three electrodes, called set B. Set A may be configured to use two of its electrodes as stimulating electrodes, with the remainder being return electrodes. Set B may be configured to have just one stimulating electrode. The controller 950 could then specify that set A deliver a stimulus phase with 3 mA current for a duration of 200 us, followed by a 400 us charge-balancing phase. This stimulus cycle could be specified to repeat at a rate of 60 cycles per second. Then, for set B, the controller 950 could specify a stimulus phase with 1 mA current for duration of 500 us, followed by a 800 us charge-balancing phase. The repetition rate for the set B stimulus cycle can be set independently of set A (e.g., at 25 cycles per second). Or, if the controller 950 was configured to match the repetition rate for set B to that of set A, for such a case the controller 950 can specify the relative start times of the stimulus cycles to be coincident in time or to be arbitrarily offset from one another by some delay interval.

In some implementations, the controller 950 can arbitrarily shape the stimulus waveform amplitude, and may do so in response to instructions from the RF pulse generator module 806. The stimulus phase may be delivered by a constant-current source or a constant-voltage source, and this type of control may generate characteristic waveforms that are static. For example, a constant current source generates a characteristic rectangular pulse in which the current waveform has a very steep rise, a constant amplitude for the duration of the stimulus, and then a very steep return to baseline. Alternatively, or additionally, the controller 950 can increase or decrease the level of current at any time during the stimulus phase and/or during the charge-balancing phase. Thus, in some implementations, the controller 950 can deliver arbitrarily shaped stimulus waveforms such as a triangular pulse, sinusoidal pulse, or Gaussian pulse for example. Similarly, the charge-balancing phase can be arbitrarily amplitude-shaped, and similarly a leading anodic pulse (prior to the stimulus phase) may also be amplitude-shaped.

As described above, the tissue stimulator 814 may include a charge balancing component 946. Generally, for constant current stimulation pulses, pulses should be charge balanced by having the amount of cathodic current should equal the amount of anodic current, which is typically called biphasic stimulation. Charge density is the amount of current times the duration it is applied, and is typically expressed in the units uC/cm². In order to avoid the irreversible electrochemical reactions such as pH change, electrode dissolution as well as tissue destruction, no net charge should appear at the electrode-electrolyte interface, and it is generally acceptable to have a charge density less than 30 uC/cm². Biphasic stimulating current pulses ensure that no net charge appears at the electrode 954 after each stimulation cycle and that the electrochemical processes are balanced to prevent net dc currents. The tissue stimulator 814 may be designed to ensure that the resulting stimulus waveform has a net zero charge. Charge balanced stimuli are thought to have minimal damaging effects on tissue by reducing or eliminating electrochemical reaction products created at the electrode-tissue interface.

A stimulus pulse may have a negative-voltage or current, called the cathodic phase of the waveform. Stimulating electrodes may have both cathodic and anodic phases at different times during the stimulus cycle. An electrode 954 that delivers a negative current with sufficient amplitude to stimulate adjacent neural tissue is called a “stimulating electrode.” During the stimulus phase, the stimulating electrode acts as a current sink. One or more additional electrodes act as a current source and these electrodes are called “return electrodes.” Return electrodes are placed elsewhere in the tissue at some distance from the stimulating electrodes. When a typical negative stimulus phase is delivered to tissue at the stimulating electrode, the return electrode has a positive stimulus phase. During the subsequent charge-balancing phase, the polarities of each electrode are reversed.

In some implementations, the charge balance component 946 uses one or more blocking capacitors placed electrically in series with the stimulating electrodes and body tissue, between the point of stimulus generation within the stimulator circuitry and the point of stimulus delivery to tissue. In this manner, a resistor-capacitor (RC) network may be formed. In a multi-electrode stimulator, one charge-balance capacitors may be used for each electrode, or a centralized capacitors may be used within the stimulator circuitry prior to the point of electrode selection. The RC network can block direct current (DC). However, the RC network can also prevent low-frequency alternating current (AC) from passing to the tissue. The frequency below which the series RC network essentially blocks signals is commonly referred to as the cutoff frequency, and in some embodiments, the design of the stimulator system may ensure that the cutoff frequency is not above the fundamental frequency of the stimulus waveform. In the example embodiment 800, the tissue stimulator 814 may have a charge-balance capacitor with a value chosen according to the measured series resistance of the electrodes and the tissue environment in which the stimulator is implanted. By selecting a specific capacitance value, the cutoff frequency of the RC network in this embodiment is at or below the fundamental frequency of the stimulus pulse.

In other implementations, the cutoff frequency may be chosen to be at or above the fundamental frequency of the stimulus, and in this scenario the stimulus waveform created prior to the charge-balance capacitor, called the drive waveform, may be designed to be non-stationary, where the envelope of the drive waveform is varied during the duration of the drive pulse. For example, in one embodiment, the initial amplitude of the drive waveform is set at an initial amplitude Vi, and the amplitude is increased during the duration of the pulse until it reaches a final value k*Vi. By changing the amplitude of the drive waveform over time, the shape of the stimulus waveform passed through the charge-balance capacitor is also modified. The shape of the stimulus waveform may be modified in this fashion to create a physiologically advantageous stimulus.

In some implementations, the tissue stimulator 814 may create a drive-waveform envelope that follows the envelope of the RF pulse received by the RX antenna 938. In this case, the RF pulse generator module 806 can directly control the envelope of the drive waveform within the tissue stimulator 814, and thus no energy storage may be required inside of the tissue stimulator 814, itself. In this implementation, the stimulator circuitry may modify the envelope of the drive waveform or may pass it directly to the charge-balance capacitor and/or electrode-selection stage.

In some implementations, the tissue stimulator 814 may deliver a single-phase drive waveform to the charge balance capacitor or it may deliver multiphase drive waveforms. In the case of a single-phase drive waveform (e.g., a negative-going rectangular pulse), this pulse includes the physiological stimulus phase, and the charge-balance capacitor is polarized (charged) during this phase. After the drive pulse is completed, the charge balancing function is performed solely by the passive discharge of the charge-balance capacitor, where is dissipates its charge through the tissue in an opposite polarity relative to the preceding stimulus. In one implementation, a resistor within the tissue stimulator 814 facilitates the discharge of the charge-balance capacitor. In some implementations, using a passive discharge phase, the capacitor may allow virtually complete discharge prior to the onset of the subsequent stimulus pulse.

In the case of multiphase drive waveforms, the tissue stimulator 814 may perform internal switching to pass negative-going or positive-going pulses (phases) to the charge-balance capacitor. These pulses may be delivered in any sequence and with varying amplitudes and waveform shapes to achieve a desired physiological effect. For example, the stimulus phase may be followed by an actively driven charge-balancing phase, and/or the stimulus phase may be preceded by an opposite phase. Preceding the stimulus with an opposite-polarity phase, for example, can have the advantage of reducing the amplitude of the stimulus phase required to excite tissue.

In some implementations, the amplitude and timing of stimulus and charge-balancing phases is controlled by the amplitude and timing of RF pulses from the RF pulse generator module 806, and in other implementations, this control may be administered internally by circuitry onboard the tissue stimulator 814, such as controller 550. In the case of onboard control, the amplitude and timing may be specified or modified by data commands delivered from the pulse generator module 806.

While the RF pulse generator module 806 and the TX antenna 810 have been described and illustrated as separate components, in some embodiments, the RF pulse generator module 806 and the TX antenna 810 may be physically located in the same housing or other packaging. Furthermore, while the RF pulse generator module 806 and the TX antenna 810 have been described and illustrated as located external to the body, in some embodiments, either or both of the RF pulse generator module 806 and the TX antenna 810 may be designed to be implanted subcutaneously. While the RF pulse generator module 806 and the TX antenna 810 have been described and illustrated as coupled via a wired connection 808, in some embodiments (e.g., where the RF pulse generator module 806 is either located externally or implanted subcutaneously), the RF pulse generator module 806 and the TX antenna 810 may be coupled via a wireless connection.

Other embodiments of positioning devices and tissue stimulation systems are within the scope of the following claims.

What is claimed is:

1. A fixation device for securing a catheter to a tissue within a body, the fixation device comprising:
a shaft defining a lumen configured to receive the catheter; and
a plurality of anchors disposed along the shaft,
wherein each anchor of the plurality of anchors comprises a plurality of protrusive elements that is biased to an extended configuration in which the plurality of protrusive elements extends radially from the shaft for engaging the tissue to secure the catheter to the tissue when the catheter is disposed within the lumen, and
wherein the plurality of protrusive elements can be adjusted from the extended configuration to a collapsed configuration in which the plurality of protrusive elements are oriented parallel to the shaft.

2. The fixation device of claim 1, wherein the lumen is sized such that the catheter can be secured to the shaft via a friction fit.

3. The fixation device of claim 1, wherein the plurality of anchors is integral with the shaft.

4. The fixation device of claim 1, wherein the plurality of anchors is axially spaced apart from each other by a distance of about 0.1 mm to about 5 mm.

5. The fixation device of claim 1, wherein each anchor of the plurality of anchors comprises a plurality of hinges at which the plurality of protrusive elements is pivotal with respect to the shaft.

6. The fixation device of claim 5, wherein the plurality of hinges comprises living hinges, elbow joints, or wire spring hinges.

7. The fixation device of claim 5, wherein the plurality of protrusive elements of each anchor of the plurality of anchors extends in a proximal direction from the plurality of hinges while the plurality of protrusive elements is arranged in the extended configuration.

8. The fixation device of claim 7, wherein the plurality of protrusive elements of each anchor of the plurality of anchors extends in a distal direction from the plurality of hinges while the plurality of protrusive elements is arranged in the collapsed configuration.

9. The fixation device of claim 1, wherein the fixation device is made of one or more materials comprising polyurethane, silicone, carbothane, and elasthane.

10. The fixation device of claim 9, wherein fixation device has a durometer of 70 A to 60D.

11. The fixation device of claim 1, wherein the plurality of protrusive elements of each anchor of the plurality of anchors is arranged about a circumference of the shaft.

12. The fixation device of claim 11, wherein the protrusive elements of the plurality of protrusive elements are equally spaced apart from each other about the circumference of the shaft.

13. The fixation device of claim 1, wherein the plurality of protrusive elements of each anchor of the plurality of anchors is disposed substantially flush with the shaft while the plurality of protrusive elements is arranged in the collapsed configuration.

14. The fixation device of claim 1, wherein the shaft defines an interior profile along the lumen that includes protrusions and recessions.

15. The fixation device of claim 14, wherein the interior profile of the shaft is complementary to an exterior profile of the catheter.

16. The fixation device of claim 1, wherein the catheter comprises a housing of an implantable tissue stimulator.

17. The fixation device of claim 1, wherein the fixation device is an implantable device.

18. The fixation device of claim 1, wherein the plurality of protrusive elements of each anchor has a length of about 0.5 mm to about 5 mm.

19. An anchoring system for securing a catheter to a tissue within a body, the anchoring system comprising:
a fixation device, comprising:
a shaft defining a lumen configured to receive the catheter, and
a plurality of anchors disposed along the shaft,
wherein each anchor of the plurality of anchors comprises a plurality of protrusive elements that is biased to an extended configuration in which the plurality of protrusive elements extends radially from the shaft for engaging the tissue to secure the catheter to the tissue when the catheter is disposed within the lumen, and
wherein the plurality of protrusive elements can be adjusted from the extended configuration to a collapsed configuration in which the plurality of protrusive elements is oriented parallel to the shaft; and
a deployment tool for assembling the fixation device with the catheter, the deployment tool comprising a tubular housing configured to carry the fixation device, and the catheter secured therein, while the plurality of protrusive elements is arranged in the collapsed configuration.

20. A method of using an anchoring system to secure a catheter to a tissue within a body, the method comprising:
placing the catheter within a fixation device carried by a tubular housing;
positioning the tubular housing, carrying the fixation device and the catheter therein, at a location adjacent to the tissue;
moving the fixation device distally out of the tubular housing at the location such that a plurality of protrusive elements of a plurality of anchors of the fixation device can expand from a collapsed configuration in which the plurality of protrusive elements is oriented parallel to a shaft of the fixation device, to an extended configuration in which the plurality of protrusive elements extends radially from the shaft to engage the tissue to secure the catheter to the tissue; and
withdrawing the tubular housing from the fixation device at the location adjacent to the tissue.
